(19) United States
(12) Patent Application Publication
(10) Pub. No.: US 2004/0267026 A1
(43) Pub. Date: Dec. 30, 2004

(54) Process for preparing 2,4,5-trisubstituted imidazoles from N-acylated alpha-amino nitriles

(76) Inventors: David Askin, Warren, NJ (US); Jaemoon Lee, Edison, NJ (US); Yong-Li Zhong, Edison, NJ (US)

Correspondence Address: Richard S. Parr, Merck & Co., Inc., Patent Dept. RY60-30, P. O. Box 2000, Rahway, NJ, 07065-0907, US

(21) Appl. No.: 10/860,916

(22) Filed: Jun. 4, 2004

Related U.S. Application Data
(60) Provisional application No. 60/476,389, filed on Jun. 6, 2003.

(51) Int. Cl. C07D 233/61

(52) U.S. Cl. 548/338.1

(57) ABSTRACT

The invention is a process for preparing an imidazole of formula I

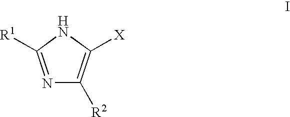

which comprises treating an N-acylated α-amino nitrile with a phosphine and a carbon tetrahalide of the formula CX₄, wherein X is Cl or Br, to form a haloimidazole of the formula

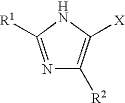

wherein

R¹ is selected from the group consisting of hydrogen, C₁₋₆alkyl, —CH₂-aryl, and aryl; and

R² is selected from the group consisting of hydrogen, C₁₋₆alkyl, —CH₂—O-aryl and aryl; and

X is selected from the group consisting of Cl and Br.

### BACKGROUND OF THE INVENTION

The synthesis of substituted-imidazoles has been given great attention due to their known biological activity and diverse medicinal uses. Several refined methods for the preparation of this class of heterocycles have been reported. Most of the synthetic methods rely on strong acid, base, high temperature or high pressure. (see Grimmett, M. R. In Comprehensive Heterocyclic Chemistry; Katritzky, A. R.; Rees, C. W.; Potts, K. T., Eds.; Pergamon Press: New York, 1984; Vol. 5, pp.457497; Grimmett, M. R. In Comprehensive Heterocyclic Chemistry II; Katritzky, A. R.; Rees, C. W.; Scriven, E. F. V.; Shinkai I., Eds.; Pergamon Press: New York, 1996; Vol. 3, pp.77-220; Tan, K. L.; Bergman, R. G.; Ellman, J. A. J. Am. Chem. Soc. 2002, 124, 13964; Laufer, S.; Wagner, G.; Kotschenreuther, D. Angew. Chem. Int. Ed. 2002, 41, 2290; Jin, Z.; Li, Z.; Huang, R. Natural Product Reports 2002, 19, 454; Groarke, M.; McKervey, A.; Nieuwenhuyzen, M. Tetrahedron Lett. 2002, 41, 1275; Sarshar, S.; Zhang, C.; Moran, E. J.; Krane, S.; Rodarte, J. C.; Benbatoul, K.; Dixon, R.; Mjialli, A. M. M. Bioorg. Med. Chem. Lett. 2002, 10, 2599; Bilodeau, M. T.; Cunningham A. M. J. Org. Chem. 1998, 63, 2800; Griffiths, G.; Hauck, M. B.; Imwinkelried, R. Kohr, J.; Roten, C. A.; Stucky, G.; Gosteli, J. ibid. 1999, 64, 8084. Rolfs, A.; Liebscher, J. ibid. 1997, 62, 3480; Shi, Y.-J; Frey, L. F.; Tschaen, D. M.; Verhoeven, T. R. Synth. Commun. 1993, 23, 2623.

Coupling reactions of carboxylic acids with amines are described in T. S. Rao; Jayaraman, K.; Revankar, G. R. Tetrahedron Lett. 1993, 34, 6189. Palladium-catalyzed coupling reactions are described in Miyaura, N.; Yamada, K.; Suginome, H.; Suzuki, A. J. Am. Chem. Soc. 1985, 107, 972; Miyaura, N.; Ishiyama, T.; Sasaki, H.; Ishikawa, M.; Satoh, M.; Suzuki, A. ibid. 1989, 111, 314; Heck, R. F. ibid. 1968, 90, 5518; Reiser, O.; Reichow, S.; de Meijere, A. Angew. Chem. Int. Ed. Engl. 1987, 26, 1277; Stille, J. K. ibid. 1986, 25, 508; Hiyama, T.; Hatanaka, Y. Pure Appl. Chem. 1994, 66, 1471.

2,4-Disubstituted-5-haloimidazoles such as 2-n-butyl4-chloro-1H-imidazole-5-carboxaldehyde, and 2-n-butyl4-chloro-5-hydroxymethylimidazole, are used, as described in U.S. Pat. No. 5,310,928 (Example 6, column 16) and U.S. Pat. No. 5,138,069 (Example 317, Part C), to prepare losartan. Larsen, et al. J. Org. Chem. 1994, 59, 6391, and Griffiths, et al., J. Org. Chem. 1999, 64, 8084, describe procedures for preparing 2-n-butyl-4-chloro-1H-imidazole-5-carboxaldehyde. Shi, et al. Synth. Commun. 1993, 23, 2623 describes a procedure for preparing 2-butyl-4(5)-chloro-5(4)-hydroxymethyl-1H-imidazole. Watson, et al., Synth. Commun. 1992, 22, 2971, describes procedures for preparing both 2-n-butyl-4-chloro-1H-imidazole-5-carboxaldehyde and 2-butyl-4(5)-chloro-5(4)-hydroxymethyl-1H-imidazole.

The present invention is a simple method for preparing 2,4-disubstituted-5-haloimidazole from N-acylated α-aminonitrile under mild conditions. A palladium-catalyzed coupling reaction is used to directly convert these halo-imidazoles to 2,4,5-trisubstituted imidazoles in high yield.

### SUMMARY OF THE INVENTION

The invention includes synthesis of 2,4,5-trisubstituted imidazole compounds, and methods of using these imidazoles to make corresponding imidazole-containing pharmaceutical compounds such as losartan, and related angiotensin H antagonists.

The following detailed description are exemplary and are intended to provide further explanation of the invention claimed.

### DETAILED DESCRIPTION OF THE INVENTION

The invention is a process for preparing an imidazole of formula I

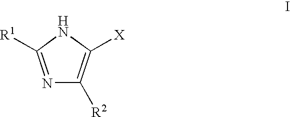

which comprises treating an N-acylated α-amino nitrile with a phosphine and a carbon tetrahalide of the formula CX₄, wherein X is Cl or Br, to form a haloimidazole of the formula

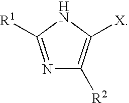

wherein

R¹ is selected from the group consisting of hydrogen, C₁₋₆alkyl, —CH₂-aryl, and aryl; and

R² is selected from the group consisting of hydrogen, C₁₋₆alkyl, —CH₂—O-aryl, and aryl.

The haloimidazole may be used to prepare losartan, described in U.S. Pat. No. 5,138,069, and other imidazole-containing angiotensin II antagonists.

In a preferred embodiment of the process, the N-Acylated α-amino nitrile is of the formula R¹C(O)NHCH(CN)(R²).

In another preferred embodiment of the process, an N-acylated α-amino nitrile of the formula R¹C(O)NHCH(CN)(R²) is treated with a phosphine and a carbon tetrahalide in a solvent at a temperature of between about 25° C. and about 55° C. for a period of between about 4 and about 20 hours, to form a 2,4-disubstituted-5-haloimidazole of the formula

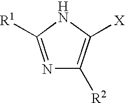

Suitable carbon tetrahalides include carbon tetrachloride and carbon tetrabromide.

Suitable phosphines include trialkylphosphines such as tributylphosphine or trioctylphosphine, and triarylphosphines such as triphenylphosphine, tritolylphosphine, or 1,2-bis(diphenylphosphino)ethane triphenylphosphine. Preferably, the phosphine is triphenylphosphine.

Suitable solvents include acetonitrile, propionitrile, dichloromethane, chloroform, carbon tetrachloride, and dichloroethane. Preferably, the solvent is acetonitrile.

The haloimidazole of the formula

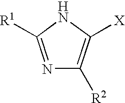

may be added to R³(BOH)₂ in the presence of a palladium-catalyzed reagent in the presence of a ligand, a base and a solvent, to provide a 2,4,5-trisubstituted-imidazole of the formula

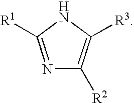

Suitable palladium-catalyzed reagents include Pd(PPh₃)₄, Pd(OAc)₂, PdCl₂, or Pd₂(dba)₃, preferably Pd₂(dba)₃.

Suitable ligands include trialkylphosphine, tributylphosphonium tetrafluoroborate, 1,4-bis(diisopropylphosphanyl)butane, and bis(diphenylphosphanyl)ferrocene, preferably tributylphosphonium tetrafluoroborate.

Suitable bases include K₃PO₄, K₂CO₃, Na₂CO₃, Cs₂CO₃, CsF, NaOH, KOH, and NaOR, wherein R is hydrogen or C₁₋₆alkyl. Preferably, the base is K₃PO₄.

Suitable solvents include toluene, dioxane, xylene, dimethylformamide, 1-methyl-2-pyrrolidinone, and dimethoxyethane, preferably toluene.

In another preferred process, a N-acylated α-amino nitrile is treated with triphenylphosphine and carbon tetrachloride or carbon tetrabromide to provide 2,4-disubstituted-5-haloimidazole, which is then subjected to a palladium-catalyzed coupling reaction to provide 2,4,5-trisubstituted imidazole.

The invention includes synthesis of 2,4,5-trisubstituted imidazole compounds and methods of using such 2,4,5-trisubstituted imidazole compounds to make losartan and related imidazole-containing angiotensin II antagonists.

Compounds prepared according to the process may have chiral centers and occur as racemic mixtures, as individual diastereomers, or as enantiomers with all isomeric forms. The scope of the present invention includes processes for preparing the individual enantiomers as well as mixtures of the enantiomers in any proportion, including racemic mixtures.

Combinations of substituents and/or variables are permissible only if such combinations result in stable compounds.

Compounds prepared according to the process of the invention are useful in preparing compounds that are useful for treating or preventing a variety of disease conditions, including hypertension and other conditions associated with angiotensin II antagonism.

Some abbreviations that may appear in this application are as follows:

| | |
|---|---|
| BOP | Benzotriazol-1-yloxytris(dimethylamino)phosphonium, |
| Dba | trans,trans-dibenzylideneacetone |
| DCC | 1,3-dicyclohexylcarbodiimide |
| DMAc | N,N-dimethylacetamide |
| DMAP | dimethylaminopyridine |
| DME | dimethoxyethane |
| DMF | dimethylformamide |
| Dppb | 1,4-bis(diisopropylphosphanyl)butane |
| Dppf | bis(diphenylphosphanyl)ferrocene |
| EDC | 1-ethyl-3-(3-dimethylaminopropyl)carbodiimide hydrochloride |
| IPAc | isopropyl acetate |
| NMP | 1-methyl-2-pyrrolidinone |
| TEA | triethylamine |
| THF | tetrahydrofuran |

Unless otherwise noted, the term “alkyl” includes both branched- and straight chain saturated aliphatic hydrocarbon groups having the specified number of carbon atoms for example, “C₁₋₆ alkyl” means an alkyl group having 1 to 6 carbon atoms, e.g., 1, 2, 3, 4, 5 or 6.” For illustration and not limitation, the alkyl may be methyl, ethyl, propyl, butyl, etc. The alkyl group may be unsubstituted or substituted with, for example, C₆₋₁₀ aryl, hydroxy, C₁₋₆ alkoxy, halogen, or amino.

Unless otherwise noted, “halogen”, as used herein, includes fluorine, chlorine, bromine, and iodine.

Unless otherwise noted, “alkoxy” means a linear or branched alkyl group of indicated number of carbon atoms attached through an oxygen bridge. “C₁₋₆ alkoxy” means any alkoxy having 1 to 6 carbon atoms, e.g., 1, 2, 3, 4, 5 or 6.

Unless otherwise noted, the term “aryl” includes a “C₁₋₆ alkoxy” means on alkoxy group having 6- to 10-membered mono- or bicyclic ring system such as phenyl, or naphthyl. The aryl ring can be unsubstituted or substituted with, for illustration and not limitation, one or more of C₁₋₆ alkyl; hydroxy; C₁₋₆ alkoxy; halogen; or amino.

It will be appreciated by those skilled in the art that compounds prepared according to the procedure of the invention may be modified to provide pharmaceutically acceptable derivatives thereof at any of the functional groups.

The pharmaceutically-acceptable salts of the compounds prepared according to the procedures described herein include those derived from pharmaceutically acceptable inorganic and organic acids such as e.g. hydrochloric, hydrobromoic, sulfuric, sulfamic, phosphoric, nitric and the like, or the quaternary ammonium salts which are formed, e.g., from inorganic or organic acids or bases. Examples of acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, nitrate, oxalate, pamoate, pectinate, persulfate, 3-phenylpropionate, picrate, pivalate, propionate, succinate, sulfate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

General Scheme I illustrates the process of the invention.

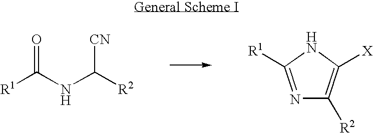

wherein

R¹ is selected from the group consisting of hydrogen, C₁₋₆alkyl, and aryl;

R² is selected from the group consisting of hydrogen, C₁₋₆alkyl, —CH₂O-aryl and aryl; and

X is selected from the group consisting of Cl or Br.

The following example illustrates the invention.

### Example 1

Preparation of N-acylated α-amino nitrile

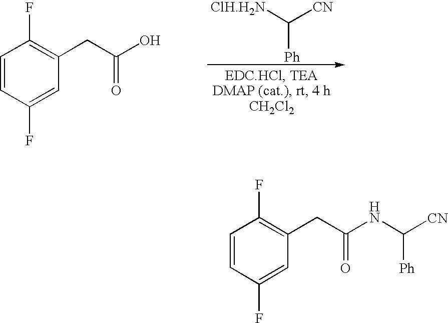

A solution of 2,5-difluorobenzylacetic acid (3.44 g, 0.0200 moles) and EDC.HCl (5.75 g, 0.0300 moles) in methylene chloride (40 mL) was treated with 2-phenylglycylnonitrile hydrochloride (3.71 g, 0.0220 moles), DMAP (0.61 g, 0.0050 moles), and triethylamine (2.23 g, 0.0220 moles) at room temperature for 4 hours. Then, 50 mL of water was added to the solution. After phase separation, the organic layer was solvent-switched to 2 N HCl aqueous solution (total volume 50 mL). The crystalline solid was filtered, washed with water (3×10 mL), and dried under vacuum with nitrogen sweep to give desired product (5.36 g, 94%) as white crystalline solid.

¹H NMR (400 MHz, CDCl₃) δ: 7.43 (brs, 5H), 7.07-6.95 (m, 3H), 6.27 (bd, J=7.9 Hz, 1H), 6.06 (d, J=7.9 Hz, 1H), 3.65 (s, 2H). ¹³C NMR (100 MHz, CDCl₃) δ: 168.3, 158.7 (d, J=250 Hz), 156.8 (d, J=240 Hz), 132.8, 129.7, 129.4, 128.0 (dd, J=20, 10 Hz), 126.8, 118.0 (d, J=20 Hz), 117.0, 116.6 (d, J=20 Hz), 116.1 (d, J=20 Hz), 44.3, 36.2. MS (m/e): 287 ([M+H]⁺, 8).

Conversion of N-acylated α-amino nitrile to 2,4-disubstituted-5-haloimidazole

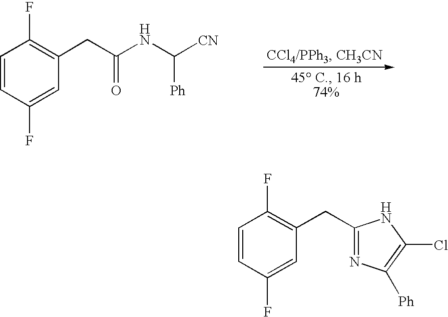

A solution of N-acylated α-amino nitrile (1.43 g, 0.00500 moles) and triphenylphosphine (3.28 g, 0.0125 moles) in acetonitrile (50 mL) was treated with carbon tetrachloride (1.93 g, 0.0125 moles) at 45° C. for 16 hours. The reaction mixture was concentrated and solvent-switched to methylene chloride (total volume 60 mL). To the solution was added 0.5 N sodium hydroxide (50 mL). The two-phase mixture was stirred at room temperature for 10 min. After phase separation, the organic layer was washed with water (2×20 mL), brine (20 mL), and concentrated. The residue was purification by fast chromatography (silica gel, hexane:EtOAc:TEA=5:1:0.05) to give desired product (1.13 g, 74%) light yellow crystalline solid.

¹H NMR (400 MHz, CDCl₃) δ: 9.98 (brs, 1H), 7.61 (bd, J=7.5 Hz, 2H), 7.41 (bt, J=7.5 Hz, 2H), 7.31 (bt, J=7.5 Hz, 1H), 7.06-6.86 (m, 3H), 4.01 (s, 2H). ¹³C NMR (100 MHz, CDCl₃) δ: 158.6 (d, J=240 Hz), 156.8 (d, J=240 Hz), 143.5, 139.0, 128.9, 128.4, 127.7, 126.8, 125.8, 125.0 (d, J=20 Hz), 117.5 (d, J=20 Hz), 116.6 (dd, J=20, 10 Hz), 115.6 (dd, J=20, 10 Hz), 28.5. MS (m/e): 305 ([M+H]⁺, 100), 307 ([M+2+H]⁺, 30).

Conversion of 2,4-disubstituted-5-haloimidazole to 2,4,5-trisubstituted-imidazole

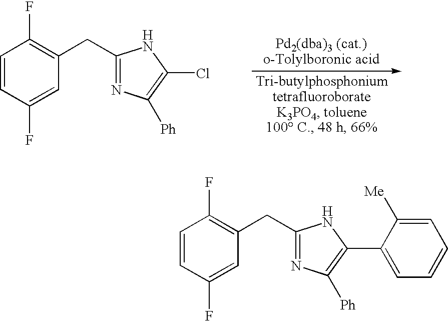

A degassed mixture of 2,4-disubstituted-5-haloimidazole (135 mg, 0.443 mmoles), Pd₂(dba)₃ (20.3 mg, 0.0222 mmoles), tri-butylphosphonium tetrafluoborate (25.7 mg, 0.0886 mmoles), potassium phosphate (282.1 mg, 1.33 mmoles), and O-tolyboronic acid (90.4 mg, 0.0665 mmoles) in toluene (5 mL) was heated at 100° C. for 48 hours. After being cooled to room temperature, the mixture was diluted with EtOAc (10 mL), then filtered. The filtrate was concentrated. The residue was purification by fast chromatography (silica gel, hexane: EtOAc:TEA=6:1:0.05) to give desired product (105 mg, 66%) light yellow crystalline solid.

¹H NMR (400 MHz, CDCl₃) o: 9.98 (brs, 1H), 7.61 (bd, J=7.5 Hz, 2H), 7.41 (bt, J=7.5 Hz, 2H), 7.31 (bt, J=7.5 Hz, 1H), 7.06-6.86 (m, 3H), 4.01 (s, 2H). ¹³C NMR (100 MHz, CDCl₃) o: 158.6 (d, J=240 Hz), 156.8 (d, J=240 Hz), 144.2, 141.5, 137.4, 130.7, 130.2, 128.6, 128.2, 128.0, 127.3 (dd, J=20, 10 Hz), 125.9, 125.8, 125.6, 117.2 (d, J=20 Hz), 116.3 (dd, J=30, 10 Hz), 114.6 (dd, J=20, 10 Hz), 27.2, 19.2. MS (m/e): 305 ([M+H]⁺, 100), 307 ([M+2+H]⁺, 30).

### Example 2

Preparation of 2-Butyl-4-chloro-5-benzyloxymethyl-1H-imidazole

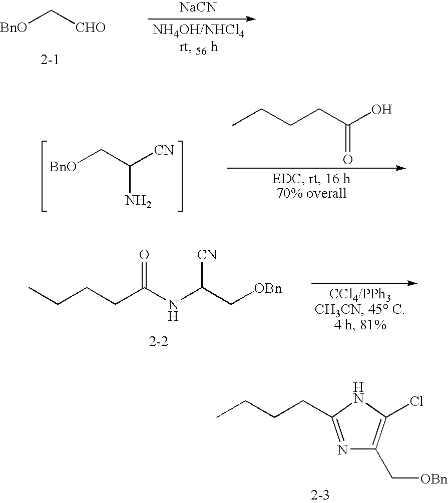

Preparation of β-Benzyloxy-α-(N-butyryl)-aminopropionitrile (2-2)

To a solution of 25% ammonium hydroxide (16 mL) was added sodium cyanide (2.04 g, 0.0417 mol), and ammonium chloride (2.63 g, 0.0492 mol) with vigorous stirring. Then, the benzyloxyacetaladehyde (5.00 g, 0.0333 mol) was added dropwise within 0.5 h at room temperature. The mixture was stirred at ambient temperature for 56 h, followed by extraction with dichloromethane (40 mL). The organic layer was washed with brine (20 mL), dried over MgSO₄, and filtered. To the filtration was added valeric acid (3.40 g, 0.0333 mol), EDC (9.58 g, 0.0500 mol), and catalytic amount DMAP (1.02 g, 0.00833 mol). The resulting mixture was stirred at room temperature for 16 h. The reaction mixture was quenched with water (50 mL). After phase separation, the organic layer was washed with 2 N HCl (50 mL), water (30 mL), brine (30 mL), dried over MgSO₄, and concentrated. The residue was purification by fast chromatography (silica gel, hexane:EtOAc=5:1) to afford β-Benzyloxy-α-(N-butyryl)-aminopropionitrile 2-2 (6.08 g, 70% overall) as colorless crystalline solid. ¹H NMR (400 MHz, CDCl₃) o: 7.45-7.18 (m, 5H), 6.28 (brs, 1H), 5.17-5.07 (m, 1H), 4.65 (d, J=12.0 Hz, 1H), 4.58 (d, J=12.0 Hz, 1H), 3.75 (dd, J=9.9, 3.3 Hz, 1H), 3.61 (dd, J=9.9, 3.8 Hz), 2.21 (t, J=7.5 Hz, 2H), 1.68-1.54 (m, 2H), 1.42-1.28 (m, 2H), 0.92 (t, J=7.4 Hz); ¹³C NMR (100 MHz, CDCl₃) 8:172.5, 136.7, 128.7 (2C), 128.3, 127.9 (2C), 117.4, 73.7, 68.9, 40.5, 35.8, 27.3, 22.2, 13.7

Preparation of 2-Butyl-4-chloro-5-benzyloxymethyl-1H-imidazole (a)

A solution of β-Benzyloxy-α-(N-butyryl)-aminopropionitrile 2-2 (781.0 mg, 3.00 mmoles) and triphenylphosphine (1.97 g, 7.50 mmoles) in acetonitrile (30 mL) was treated with carbon tetrachloride (1.74 mL, 7.50 mmoles) at 45° C. for 4 hours. The reaction mixture was concentrated and solvent-switched to methylene chloride (total volume 50 mL). To the solution was added 0.5 N sodium hydroxide (30 mL). The two-phase mixture was stirred at room temperature for 10 min. After phase separation, the organic layer was washed with water (2×15 mL), brine (15 mL), and concentrated. The residue was purification by fast chromatography (silica gel, hexane:EtOAc=5:1, 4:1) to give 2-butyl-4-chloro-5-benzyloxymethyl-1H-imidazole 2-3 (675.0 mg, 81%). ¹H NMR (400 MHz, CDCl₃) 6:11.45 (brs, 1H), 7.34-7.27 (m, 5H), 4.51 (s, 2H), 2.59 (t, J=7.6 Hz, 2H), 1.65-1.57 (m, 2H), 1.34-1.25 (m, 2H), 0.86 (t, J=7.3 Hz, 3H); ¹³C NMR (100 MHz, CDCl₃) δ: 148.4, 137.5, 128.4 (2 C), 128.0, 127.9 (2 C), 127.3, 120.9, 71.9, 60.9, 30.3, 28.3, 22.2, 13.7.

### Example 3

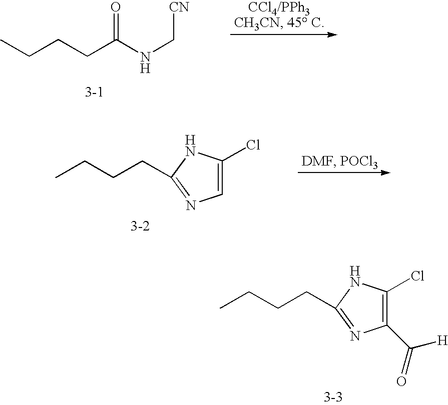

Compound 3-3 is prepared from 3-2 according to procedures described in European Patent 743306 and U.S. Pat. No. 5,508,425. 3-3 is used, in accordance with Scheme 4a and Larsen, et al., J. Org. Chem. 1994, 59, 6391, to make losartan.

### Example 4

Preparation of 2-Butyl-4-chloro-5-hydroxymethyl-1H-imidazole (4-1), and subsequent Preparation of Losartan

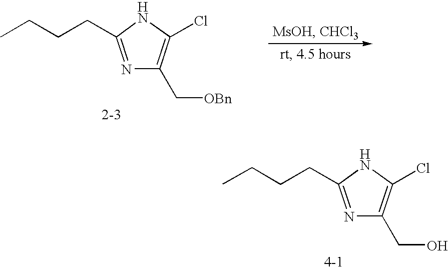

A mixture of 2-butyl-4-chloro-5-benzyloxymethyl-1H-imidazole 2-3 (139.4 mg, 0.500 mmol) and methanesulfonic acid (1.25 mL) in chloroform (2.8 mL) was stirred at room temperature for 1 h. The reaction mixture was poured onto ice (5 g). The resulting solution was neutralized by 5 N sodium hydroxide to adjust to pH=10. The solution was extracted with MTBE (2×5 mL). The aqueous layer was extracted with n-butanol (3×5 mL). The combined extractions from n-butanol were concentrated and solvented-switched to toluene. The crystalline solid was filtered off, washed with cold water, hexane/MTBE (4:1), dried under vacuum to give 2-Butyl-4-chloro-5-hydroxymethyl-1H-imidazole 4-1 (84.3 mg, 90%). LC/MS (m/z): 189.1 [(M+H)+].

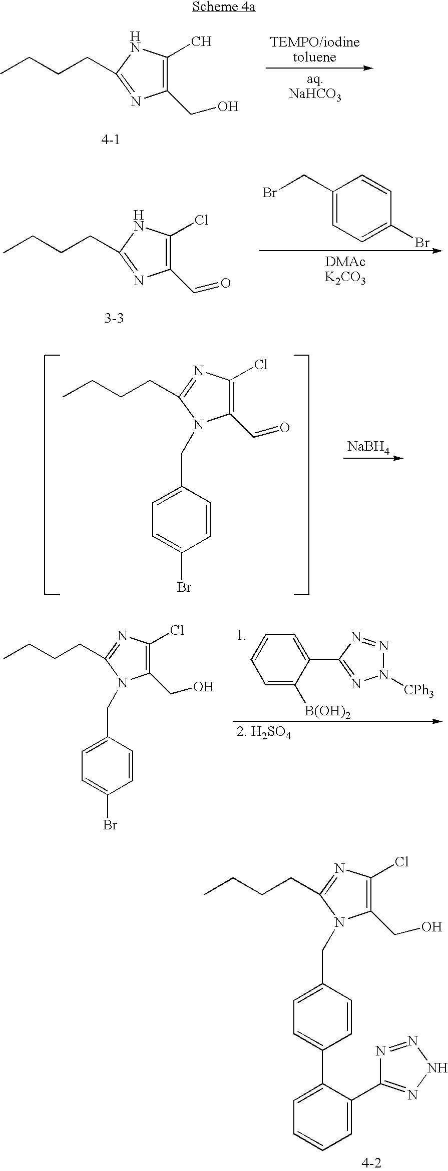

Following the procedure outlined in Scheme 4a and described in Miller et al., Organic Letters 2003 Vol. 5, No. 3, pp. 285-287, 2-Butyl-4-chloro-5-hydroxymethyl-1H-imidazole 4-1 is oxidized to compound 3-3. 3-3 is used to make losartan following Larsen, et al., J. Org. Chem. 1994, 59, 6391.

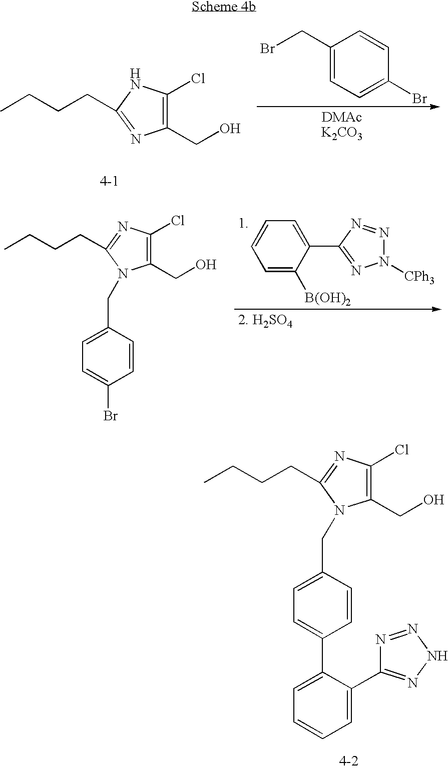

Alternatively, following the procedure outlined in Scheme 4b and described in Larsen et al., J. Org. Chem. 1994, 59, 6391, 4-1 is used to make losartan.

What is claimed is:

1. A process for preparing an imidazole of formula I 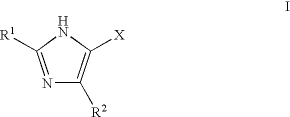
which comprises treating an N-acylated α-amino nitrile with a phosphine and a carbon tetrahalide of the formula CX₄, wherein X is Cl or Br, to form a haloimidazole of the formula 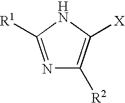
wherein
R¹ is selected from the group consisting of hydrogen, C₁₋₆alkyl, —CH₂-aryl, and aryl; and
R² is selected from the group consisting of hydrogen, C₁₋₆alkyl, —CH₂—O-aryl and aryl; and
X is selected from the group consisting of Cl and Br.

2. A process of claim 1, wherein the N-acylated α-amino nitrile is of the formula R¹C(O)NHCH(CN)(R²).

3. A process of claim 1, wherein the N-acylated α-amino nitrile is treated with a phosphine and a carbon tetrahalide selected from the group consisting of carbon tetrachloride and carbon tetrabromide, in a solvent at a temperature of between about 25° C. and about 55° C. for a period of between about 4 and about 20 hours, to form a 2,4-disubstituted-5-haloimidazole of the formula

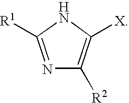

4. A process of claim 3, wherein the phosphine is a trialkylphosphine or a triarylphosphine.

5. A process of claim 4, wherein the trialkylphosphine is selected from the group consisting of tributylphosphine and trioctylphosphine, and the triarylphosphine is selected from the group consisting of triphenylphosphine, tritolylphosphine, and 1,2-bis(diphenylphosphino)ethane triphenylphosphine.

6. A process of claim 5, wherein the triarylphosphine is triphenylphosphine.

7. A process of claim 3, wherein the solvent is selected from the group consisting of acetonitrile, propionitrile, dichloromethane, chloroform, and dichloroethane.

8. A process of claim 7, wherein the solvent is acetonitrile.

9. A process of claim 1, wherein a haloimidazole of the formula 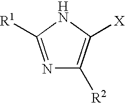
is added to R³(BOH)₂ in the presence of a palladium-catalyzed reagent, a ligand, a base and a solvent, to provide a 2,4,5-trisubstituted-imidazole of the formula

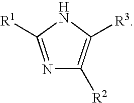

10. A process of claim 9, wherein the palladium-catalyzed reagent is selected from the group consisting of Pd(PPh₃)₄, Pd(OAc)₂, PdCl₂, or Pd₂(dba)₃.

11. A process of claim 9, wherein the palladium-catalyzed reagent is Pd₂(dba)₃.

12. A process of claim 9, wherein the ligand is selected from the group consisting of trialkylphosphine, tributylphosphonium, 1,4-bis(diisopropylphosphanyl)butane, and bis(diphenylphosphanyl)ferrocene.

13. A process of claim 12, wherein the ligand is tributylphosphonium.

14. A process of claim 9, wherein the base is selected from the group consisting of K₃PO₄, K₂CO₃, Na₂CO₃, Cs₂CO₃, CsF, NaOH, KOH, and NaOR, wherein R is hydrogen or C₁₋₆alkyl.

15. A process of claim 14, wherein the base is K₃PO₄.

16. A process of claim 9, wherein the solvent is selected from the group consisting of toluene, dioxane, xylene, dimethylformamide, 1-methyl-2-pyrrolidinone, and dimethoxyethane.

17. A process of claim 16, wherein the solvent is toluene.
